(19) United States
(12) Patent Application Publication
(10) Pub. No.: US 2008/0171791 A1
(43) Pub. Date: Jul. 17, 2008

(54) Compositions and Method for Losing Weight

(75) Inventor: Alfred B. Ordman, Monticello, WI (US)

Correspondence Address: Patricia McQueeney;MDR Fitness Corp., 14101 NW 4th Street, Sunrise, FL, 33325, US

(73) Assignee: MDR Fitness Corp., Sunrise, FL (US)

(21) Appl. No.: 12/013,105

(22) Filed: Jan. 11, 2008

Related U.S. Application Data
(60) Provisional application No. 60/879,995, filed on Jan. 11, 2007.

(51) Int. Cl. A61K 31/201; A61K 31/198

(52) U.S. Cl. 514/560; 514/561

(57) ABSTRACT

Methods and compositions for assisting in human weight loss. In a preferred embodiment, the administration of branched-chain amino acids and fatty acids to a subject at a level that provides approximately a two and a half percent (2.5%) reduction in weight over a one-week period.

### CROSS-REFERENCE TO RELATED APPLICATIONS

Not Applicable.

### STATEMENT REGARDING FEDERALLY SPONSORED RESEARCH OR DEVELOPMENT

Not Applicable.

### NAMES OF PARTIES TO JOINT RESEARCH AGREEMENT

Not Applicable.

### INCORPORATION-BY-REFERENCE OF MATERIAL SUBMITTED ON COMPACT DISC

Not Applicable.

### BACKGROUND

This invention relates to a dosage form, a method to manufacture the dosage form and method to use the dosage form to assist people in losing weight. More particularly, the dosage form contains effective quantities of one or more branched-chain amino acids and one or more unsaturated fatty acids. For a person weighing 150 pounds, those quantities are between approximately one-half (0.5) gram (g) and approximately forty (40) grams (g) of the amino acids and between approximately one-half (0.5) gram (g) and approximately forty (40) grams (g) of the fatty acids. Those quantities may be provided once a day or several times per day in smaller portions that total the effective quantities.

Throughout this application, the term amino acid refers to alpha-amino acids.

Performing an internet search powered by the Google® search engine for the phrase “weight loss” yields 160 million results. For example, the website whichdietpill.com lists the top five weight loss diet pills¹ as determined from a combination of customer results, personal success, customer satisfaction, company reputation, best selling product, and highest reorder rate. The number one listed product at the time this application was drafted is a capsule containing a combination of Hoodia gordonii, magnesium, Garcinia cambogia extract, green tea and a proprietary blend. Hoodia gordonii is an African cactus. It is believed that the active ingredient in Hoodia gordonii is a steroidal glycoside, which is believed to communicate to the brain that a sufficient blood sugar level exists, thereby suppressing appetite. No peer-reviewed literature is provided indicating either that this method works or that this alleged mechanism of action has been demonstrated ¹Applicant uses the term “pill” loosely, as this dosage form is almost obsolete.

The second product listed in the whichdietpill.com list of top five weight loss pills uses white kidney bean extract to purportedly neutralize a digestive enzyme (α-amylase) that converts starch into glucose and then fat. The use of this product allows carbohydrates to allegedly pass through the digestive tract without being processed into fat. Once again, no scientific literature is provided nor have double-blind studies been performed to demonstrate the effectiveness of this product.

The third of the top five weight loss pills is a combination of L-carnitine, green tea, Camellia sinensis, α-lipoic acid, biotin, Piper nigrum and additional excipients. This combination allegedly acts as an appetite suppressant, although a clear mechanism is not provided.

The above three examples are typical of currently advertised, non-prescription weight loss supplements. No weight loss product has been found that is based on published peer-reviewed scientific literature, nor have any current weight loss products been found that have been shown to be effective in published peer-reviewed scientific and medical research.

In the May 12, 2006 issue of Science (Science 312:927-930 (2006)), the teachings of which are incorporated herein in their entirety, researchers demonstrated that branched-chain amino acids (“BCAAs”) play a part in the mammalian target of rapamycin (mTOR) signaling pathway. Signaling by the mTOR pathway plays a role in the brain mechanisms that respond to nutrient availability and regulate energy balance. In particular, the researchers administered L-leucine near rats' arcuate nucleus of the hypothalamus. The administration caused a decrease in the rats' food intake approximately four hours after administration that lasted for a period of 24 hours. The decrease in food intake was accompanied by weight loss. Administration of L-valine, however, did not cause the same effect in that study.

In the study by T. B. Seaton et al., “Thermic effects of medium-chain and long-chain triglycerides in man.” Am J Clin Nutr 44:630-634(1986), the teachings of which are incorporated herein in their entirety, medium-chain triglycerides (MCTs) have been shown to have potential weight loss benefits. In this reported study, seven (7) men had higher metabolic rates after consuming Medium Chain-length Triglycerides (“MCTs”). After reviewing this study, the present inventor theorized that these men might lose weight after consuming MCTs if their energy (i.e., food) intake stayed constant.

Finally, a study published by the American Society for Nutrition (J. Nutr. 136: 1779-85 (2006)), the teachings of which are incorporated herein in their entirety, revealed that rodents fed diets high in unsaturated fat experienced less weight gain than rodents fed diets high in saturated fat. The researchers found that the optimal activity of the nuclear hormone receptor (PPAR) that controls lipid oxidation, adipocyte differentiation, glucose and lipid storage, and inflammation was found to be activated by fatty acids having a 16 to 20 carbon chain length and several double bonds in the chain. The researchers concluded that linoleic acid (C18:2) and arachidonic acid (C20:4) were more effective in the suppression of lipogenic genes than oleic acid (C18-1).

Though many over-the-counter supplements claim to cause weight loss, the world is still suffering from an obesity epidemic. In contrast to existing options, the ingredients listed in the present invention have been experimentally tested and the mechanism of operation of each ingredient has been proven in peer-reviewed scientific journals. To date, however, no one has discovered 1) the effect of the combined ingredients in humans, 2) the superior weight loss benefit provided to humans by the combination, or 3) the dosages of the ingredients necessary to achieve the superior weight loss in humans. All three of these discoveries are disclosed in the present invention.

### BRIEF SUMMARY

It is therefore an object of this invention to disclose a combination of ingredients that can assist humans in losing weight.

It is an object of the invention to provide a dosage form for delivery of those ingredients.

It is also an object of this invention to provide a method of using this dosage form to lose weight.

Finally, it is an object of this invention to provide a method of making this dosage form.

These and other objects and features will be more readily understood from a consideration of the following detailed description.

### DETAILED DESCRIPTION

As a result of extensive experience in the nutrition field, the present inventor has discovered that administration of branched-chain amino acids and unsaturated fatty acids shortly before or concurrent with a meal helps to suppress an individual's appetite and provides rapid weight loss. In addition, upon discontinuance of the administration, the weight loss candidate is not subject to the rebound effect encountered with many other popular diets.

Branched-chain amino acids include leucine, isoleucine, valine or any combination thereof. These amino acids are found in proteins of all living organisms. The recommended daily allowance (RDA) of leucine for adults is approximately 16 milligrams (mg) per kilogram (kg) of bodyweight. The RDA of isoleucine for adults is approximately 10-12 mg/kg of bodyweight and that of valine for adults is approximately 14 mg/kg of bodyweight. The chemical formulae for the three are as follows:

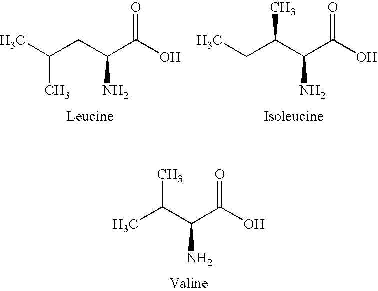

All three branched-chain amino acids have been used by body builders for years to help with muscle recovery, although little data is available to show that this use actually improves athletic performance. All three are commercially available as capsules, tablets and powders.

Leucine, which is slightly soluble in water, is isolated into white crystals or a crystalline powder. As with any water soluble supplement, excess quantities will not normally damage the body because the excess amount that is not utilized by the body is excreted by urine.

As an aside, the long-term administration of large quantities of leucine has been linked to pellagra, a disease that is associated with niacin and protein deficiencies. Persons utilizing the teachings of the present invention should monitor themselves for the development of such deficiencies. However, commercially available leucine supplements in the range necessary for this invention contain no such warnings. Furthermore, the servings of meat at restaurants release similar levels of leucine as they are digested. For example, body protein contains approximately eight percent (8%) leucine. Therefore, a quarter-pound hamburger contains approximately 9 g of leucine (0.25 lbs×454 g/lbs×0.08).

Isoleucine, which is not soluble in water, is isolated into a white to very slightly yellow powder. Due to its stereochemistry, four stereoisomers of isoleucine are possible. However, the isoleucine found in nature is (2S, 3S)-2-amino-3-methylpentanoic acid. Although not water soluble, health risks are not associated with high doses of isoleucine, unless a person suffers from kidney or liver disease. However, reports of increased urination have been received from people who ingest higher amounts of isoleucine.

Like leucine, valine is water soluble. Valine is listed as one of 599 additives to cigarettes in the 1994 report released by the top five cigarette companies. Similarly to most cigarette additives, its use or purpose is unknown. People consuming large quantities of valine have reported occurrences of both hallucinations and crawling sensations on the skin.

Recommended dosages of the branched chain amino acids in accordance with the present invention range from approximately one hundred milligrams (mg) to approximately eight (8) g per 30 pounds body weight per day. For example, a person weighing 150 pounds at the beginning of the claimed weight loss method ingests approximately five hundred (500) mg to approximately forty (40) g of leucine per day. As the person loses weight, the dosage amount may be reduced to maintain a dosage range between approximately 100 mg to approximately 8 g leucine per 30 pounds body weight. In another embodiment, a person weighing 150 pounds at the beginning of the claimed weight loss method may ingest approximately five hundred (500) mg to approximately forty (40) g of a combination of leucine, isoleucine and valine and use the same combination to maintain the dosage. One or ordinary skill in the art would recognize that many different combinations of amino acids may be utilized in the teachings of the present invention.

Unsaturated fatty acids are carboxylic acids having the chemical formula RCOOH in which the “R” represents an unsaturated aliphatic tail. Examples of unsaturated fatty acids include crotonic acid (4:1), myristoleic acid (14:1), palmitoleic acid (16:1), oleic acid (aka 9-octadecenoic acid) (18:1), vaccenic acid (aka 11-octadecenoic acid) (18:1), linoleic acid (18:2), linolenic acid (18:3), gadoleic acid (20:1), arachidonic acid (20:4), eicosapentaenoic acid (20:5), erucic acid (22:1), and docosahexaenoic acid (22:6), to name a few. Unsaturated fatty acids are divided into two groups, mono-unsaturated and poly-unsaturated. Mono-unsaturated fatty acids have one double bond in the aliphatic tail, whereas poly-unsaturated fatty acids have more than one. Unsaturated fatty acids can be found in day-to-day items such as algae, as well as fish, olive, camellia, sunflower, canola, almond, grapeseed, peanut, flaxseed, fish, walnut, soybean, macadamia nut and corn oils, to name a few.

Recommended dosages of the unsaturated fatty acids in accordance with the present invention range from approximately one hundred milligrams (mg) to approximately eight (8) g per 30 pounds body weight per day. For example, a person weighing 150 pounds at the beginning of the claimed weight loss method would ingest approximately five hundred (500) mg to approximately forty (40) g of oleic acid per day. As the person loses weight, the dosage amount may be reduced to maintain a dosage range between approximately 100 mg to approximately 8 g oleic acid per 30 pounds body weight. In another embodiment, a person weighing 150 pounds at the beginning of the claimed weight loss method may ingest approximately five hundred (500) mg to approximately forty (40) g of a combination of oleic acid, linoleic acid and docosahexaenoic acid and use the same combination to maintain the dosage. One or ordinary skill in the art would recognize that many different combinations of unsaturated fatty acids may be utilized in the teachings of the present invention.

In the original experiment, the present inventor tested a combination of coconut oil and L-leucine. As stated in the Background, Medium-Chain Triglycerides (MCTs) such as coconut oil have been advertised to have potential weight loss benefits. Am J Clin Nutr 44:630-634(1986). The fatty acid breakdown of coconut oil is 85.2% saturated fatty acids, 6.6% mono-unsaturated fatty acids, and 1.7% polyunsaturated fatty acids. In the experiment the combination of coconut oil and L-leucine was provided to two (2) men and one (1) woman twice a day for two weeks. None of the subjects experienced any weight loss.

Based upon his extensive experience and review of a variety of current research publications, the inventor substituted olive oil for the coconut oil used in the original experiment. Olive oil contains approximately 70% oleic acid. The fatty acid breakdown of olive oil is 14.0% saturated fatty acids, 69.7% mono-unsaturated fatty acids and 11.2% polyunsaturated fatty acids. As will be discussed in connection with the following examples, this combination proved successful. It is theorized that the elevated percentage of monounsaturated fatty acids in olive oil provided the necessary signaling to both brown adipose tissue and subcutaneous fat.

The present invention is designed to provide adequate dosages to change the way the human brain works as described and demonstrated in animals in the cited peer-reviewed references. Leucine and oleic acid are absorbed intact from the digestive tract and are capable of crossing the blood-brain barrier. Leucine is an essential amino acid, which means that humans cannot synthesize it and must obtain it from the diet. In order to synthesize proteins in the body, including the brain, it must be absorbed into the blood and pass to every cell in the body.

A phenomenon common after discontinuance of many diets is known as the rebound effect. This phenomenon, unwanted by most people seeking to lose weight, is likely to occur because adipose tissue has a set point, so that the cells in the adipose tissue store additional fat causing the subject to return to the weight previous to dieting. After discontinuing administration of the present invention, there is no immediate rebound effect because fat cells maintain the storage of fat at the level present at the end of consuming the dosage, rather than rebounding and restoring fat to the level present before the invention was applied. The inventor demonstrated this lack of a rebound effect after use of this invention. In that experiment, the inventor lost 6 pounds over 9 days. After discontinuing use of the invention, the inventor did not regain any of the lost weight for a period of 2 months. Assuming other subjects using the invention do not deliberately behave in a manner that will cause them to regain the lost weight, the subjects will not rapidly regain the weight lost during administration of the present invention after discontinuing its usage.

An additional embodiment of the invention relates to the administration of the dosage form, either alone or in conjunction with pharmaceutically or nutritionally acceptable carriers. The dosage form may include a separate delivery mechanism for each active ingredient. For example, taking an individual capsule of olive oil with a glass of orange juice containing powdered leucine would be included within the teachings of the present invention.

In addition to the active ingredients, the delivery mechanisms may contain suitable pharmaceutically- or nutritionally-acceptable carriers comprising excipients and auxiliaries which facilitate processing of the active compounds into preparations which can be used pharmaceutically or nutritionally. For example, the active ingredients may be incorporated into a butter-type spread or a chocolate-flavored candy. Further details on techniques for pharmaceutical formulation and administration may be found in the latest edition of Remington's Pharmaceutical Sciences (Maack Publishing Co., Easton, Pa.).

The active ingredients may be administered alone or in combination with one or more agents, such as stabilizing compounds, which may be administered in any sterile, biocompatible pharmaceutical or nutritional carrier. The compositions may be administered to a patient alone, or in combination with other agents, drugs or hormones.

The compositions utilized in this invention may be administered by any number of routes including, but not limited to, oral, sublingual, intravenous, intramuscular, intraarterial, intramedullary, intrathecal, intraventricular, subcutaneous, intraperitoneal, intranasal, intraocular, enteral, sublingual, or rectal means. At the present time, it is not believed that topical or transdermal administration will prove effective. However, as topical and/or transdermal delivery mechanisms improve, such administration may also become a suitable option. For example, many patented transdermal delivery mechanisms that eliminate the barrier effect of the skin may be suitable for use with the present invention.

Pharmaceutical compositions for oral administration can be formulated using pharmaceutically acceptable carriers well known in the art that enable provision of the dosages of active ingredients previously discussed herein. Such carriers enable the pharmaceutical compositions to be formulated as tablets, pills, dissolving strips, dragees, capsules, dragees, liquids, gels, syrups, slurries, suspensions, lozenges, chews, solid solutions including powdered drink and/or effervescent formulations, liposomes, films, ovules, and sprays and the like, for ingestion by the patient. Any of these dosage forms may include a liposome delivery mechanism in which the ingredients are encapsulated into liposomes. Additional ingredients to improve the taste and stability of the formulation may also be included.

Pharmaceutical preparations for oral use can be obtained through combination of the active compounds with excipients, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, sorbitol, sucralose and fructose; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

One or more of the active ingredients, the branched chain amino acids and the unsaturated fatty acids, may also be provided in a salt form, which may be formed with many acids, including but not limited to, hydrochloric, sulfuric, acetic, lactic, tartaric, malic, succinic, etc. For example, sodium oleate may be used in place of oleic acid. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

The following examples are provided to illustrate the present invention and to assist one of ordinary skill in the art in making and using the same. These examples are not intended in any way to limit the scope of the invention as defined by the appended claims.

### EXAMPLE 1

One embodiment of the present invention involved the combination one (1) cup (125 g) leucine with one (1) cup (16 tablespoons) extra virgin olive oil. The resulting mixture was stirred until the leucine dissolved. The mixture was refrigerated until it formed a butter-textured consistency, at which point it was ready for ingestion.

One subject took one tablespoon in the morning upon awakening and a second tablespoon before dinner. The subject lost six (6) pounds within nine (9) days, with no change in eating and exercise behavior. The subject's weight remained at the new weight for a month after discontinuing administration of the mixture.

### EXAMPLE 2

A second embodiment of the present invention involves the concurrent administration of olive oil capsules, which are commercially available, and branched-chain amino acid capsules containing a combination of L-leucine, L-isoleucine and L-valine, also commercially available. The combination will be taken twice a day for two weeks. It is anticipated that the subject will lose approximately 2.5% (two and a half percent) of his/her body weight after a one week period. Furthermore, it is theorized that the subject will not regain this weight after discontinuing administration of the present invention.

The instant invention has been disclosed in its most practical and preferred embodiments. However, the invention is not restricted to the illustrative examples described above, but may be modified within the scope of the following claims without departing from the intended scope of the invention.

I claim:

1. A dosage form comprising one or more branched-chain amino acids and one or more unsaturated fatty acids.

2. The dosage form of claim 1, in which the branched-chain amino acid is leucine.

3. The dosage form of claim 1, in which the unsaturated fatty acid is oleic acid.

4. The dosage form of claim, in which the unsaturated fatty acid is docosahexaenoic acid.

5. The dosage form of claim 1, in which the unsaturated fatty acid is eicosapentaenoic acid.

6. The dosage form of claim 1, in which the unsaturated fatty acid is a combination of docosahexaenoic acid and eicosapentaenoic acid.

7. The dosage form of claim 1, in which the unsaturated fatty acid is Omega-3 fatty acid.

8. The dosage form of claim 1, in which the branched-chain amino acid is leucine and wherein the unsaturated fatty acids are contained in extra virgin olive oil.

9. A method of assisting a human having a predetermined body weight to lose weight, the method comprising: administering at least daily the dosage form of claim 1, in which the at least daily administration occurs for a specified period of time.

10. The method of claim 9, in which the at least daily administration of the dosage form comprises providing daily administration of not less than approximately one hundred (100) milligrams (mg) of branched-chain amino acid per thirty (30) pounds of predetermined body weight and not less than approximately one hundred (100) milligrams (mg) of unsaturated fatty acid per thirty (30) pounds of predetermined body weight.

11. The method of claim 10, in which the daily administration does not exceed approximately eight (8) grams of branched-chain amino acid per thirty (30) pounds of predetermined body weight and not more than approximately eight (8) grams of unsaturated fatty acid per thirty (30) pounds predetermined body weight.

12. The method of claim 9, in which the at least daily administration comprises administering a portion of or the entire dosage form approximately zero (0) to approximately three (3) hours prior to a meal.

13. The method of claim 12, in which the portion of the dosage form is determined based upon the number of meals eaten in one day.

14. The method of claim 9, in which the specified period of time is based upon obtaining the desired amount of weight loss.

15. The method of claim 9, further comprising discontinuance of the at least daily administration of the dosage form after the specified period of time without suffering a rebound effect.

16. A method of making the dosage form of claim 1, the method comprising:
providing approximately equal quantities of the branched-chain amino acid and the unsaturated fatty acid.

17. The method of claim 16, further comprising mixing the approximately equal quantities of the branched-chain amino acid and the unsaturated fatty acid to produce a mixture.

18. The method of claim 17, further comprising placing the mixture in a dosage delivery formulation.

19. The method of claim 17, in which the unsaturated fatty acid is in a liquid state and the branched-chain amino acid is in a solid state, further comprising dissolving the branched-chain amino acid in the unsaturated fatty acid to produce an admixture.

20. The method of claim 19, further comprising placing the admixture in a dosage delivery formulation.
